(19) United States
(12) Patent Application Publication
(10) Pub. No.: US 2020/0090812 A1
(43) Pub. Date: Mar. 19, 2020

(54) MACHINE LEARNING FOR MEASURING AND ANALYZING THERAPEUTICS

(71) Applicant: Smart Score Technologies, LLC, Mapleton, UT (US)

(72) Inventors: Brandon E. Condie, Mapleton, UT (US); Gary L. Norton, Mapleton, UT (US)

(73) Assignee: Smart Score Technologies, LLC, Mapleton, UT (US)

(21) Appl. No.: 16/513,224

(22) Filed: Jul. 16, 2019

Related U.S. Application Data
(60) Provisional application No. 62/698,777, filed on Jul. 16, 2018.

(51) Int. Cl. G16H 50/30; G06N 3/04; G06N 3/08; G16H 50/20

(52) U.S. Cl. CPC G16H 50/30; G16H 50/20; G06N 3/08; G06N 3/04

(57) ABSTRACT

The disclosure extends to systems and methods for measuring and recording therapeutics. A method includes receiving a plurality of user ratings responsive to health metric categories and receiving a journal entry comprising one or more of text, images, or videos. The method includes calculating an overall health score for the user based at least in part on the plurality of user ratings. The method includes storing a plurality of overall health scores for the user over time. The method includes assessing the plurality of overall health scores for the user over time to identify a correlation between at least one health metric category and a positive or negative change in the overall health score for the user. The method includes personalizing the health metric categories provided to the user based on which health metric categories have the greatest correlation with a positive or negative change in the overall health score for the user.

### CROSS-REFERENCE TO RELATED APPLICATIONS

This application claims priority to U.S. Provisional Patent Application Ser. No. 62/698,777 filed Jul. 16, 2018 titled “SYSTEMS AND METHODS FOR MEASURING AND RECORDING THERAPEUTICS,” which is incorporated herein by reference in its entirety, including but not limited to those portions that specifically appear hereinafter, the incorporation by reference being made with the following exception: In the event that any portion of the above-referenced application is inconsistent with this application, this application supersedes the above-referenced application.

### BACKGROUND

Mental health includes a person's emotional, psychological, and social wellbeing. Mental health impacts how a person thinks, feels, and behaves. Mental health can determine how a person handles stress, relates to others, and makes choices. Mental health is an important aspect of a person's overall health at every stage of the person's life, from childhood and adolescence through adulthood.

Over the course of a person's life, an individual may experience mental health struggles and issues that can impact the person's thinking, mood, and behavior. Many factors may contribute to a person's mental health, including biological factors such as genes or brain chemistry, life experiences such as trauma or abuse, and family history or genetic dispositions.

Mental health issues can become very serious and can have negative consequences in a person's life and happiness. Mental health issues may sometimes be predicted based on early warning signs.

In the United States, one in five people will be impacted by a mental health issue during a given year. In 2017, there were 47,000 suicides in the United States and an addition 1.4 million suicide attempts. Together, these issues account for an estimated $250 billion in healthcare and other costs, and a drain on work productivity and the economy. The increase in mental health issues has created an emerging market that many have tried to address. Self-help and self-improvement is a self-guided improvement—economically, intellectually, or emotionally—often with a substantial psychological basis. Many different self-help groups and programs exist, each with its own focus, techniques, associated beliefs, proponents and in some cases, leaders. Concepts and terms originating in self-help culture and twelve-step culture, such as recovery, dysfunctional families, and codependency have become firmly integrated in mainstream language.

What is needed are more effective systems and methods for helping individuals control their overall health and wellbeing by utilizing measurements and recording therapeutics, which may improve performance and increase efficiency. In light of the foregoing, disclosed herein are systems, methods, and devices for scoring, tracking, and predicting mental health. Embodiments of the disclosure permit users to navigate and analyze their own mental health progress and take initiatives to improve their overall wellbeing.

### BRIEF DESCRIPTION OF THE DRAWINGS

Non-limiting and non-exhaustive implementations of the present disclosure are described with reference to the following figures, wherein like reference numerals refer to like or similar parts throughout the various views unless otherwise specified. Advantages of the present disclosure will become better understood with regard to the following description and accompanying drawings where:

FIG. 1 is a schematic diagram of a system for scoring, tracking, and predicting the mental health and overall wellbeing of a user, according to embodiments of the disclosure;

FIG. 2 is a schematic diagram of a process flow for scoring, tracking, and predicting the mental health and overall wellbeing of a user, according to embodiments of the disclosure;

FIG. 3 illustrates an example screenshot of a user interface providing a means for a user to enter user ratings responsive to health metric categories;

FIG. 4 illustrates an example screenshot of a user interface providing a means for a user to enter an indication the user performed a booster action;

FIG. 5 illustrates an example screenshot of a user interface providing an overall health score to a user;

FIG. 6 illustrates an example screenshot of a user interface providing a means for a user to view and enter journal entries;

FIG. 7 illustrates an example screenshot of a progress summary for a user's mental health and overall wellbeing;

FIG. 8 is a schematic diagram of a long short-term memory recurrent neural network;

FIG. 9 is a schematic diagram of a training phase for a variational autoencoder network;

FIG. 10 is a schematic diagram of a process flow assessing user inputs with a neural network to predict positive and negative changes in the user's overall health score, according to embodiments of the disclosure; and

FIG. 11 illustrates a schematic diagram of an example computing device.

### DETAILED DESCRIPTION

Systems, methods, and devices for scoring, tracking, and predicting mental health and wellbeing are disclosed. An embodiment of the disclosure provides interactive technology that can be easily navigated by a user to analyze their own wellbeing and identify which elements of the user's behavior and habits are in alignment, and which can be improved. An embodiment of the disclosure permits a user to construct a personalized support system. The user may select trusted contacts and configure triggers for when each contact should be notified. An embodiment of the disclosure includes a neural network for analyzing scores, statistics, psychology, behavior analytics, and history to predict changes to the user's wellbeing and mental health.

Systems, methods, and devices of the disclosure provide means to assist users in taking control of their mental health and overall wellbeing. An embodiment of the disclosure is an application or user interface that combines multiple technologies and information sets to predict future highs and lows in a user's wellbeing. Mental health issues have always been present but have mostly been ignored and overlooked by a large portion of the population. Traditionally, there is a stigma associated with mental health that causes many people to be ashamed and closed-off when attempting to improve their mental health and overall wellbeing. In recent years, there has been a shift in public awareness of mental health issues and a new willingness to speak about mental health through open discourse. However, there are few tools that provide means for people to navigate their own mental health and the mental health of others.

The foundation of many mental health treatment programs relies on a grassroots effort to support individuals. However, this is difficult because many people feel timid discussing mental health issues with others. Loved ones may feel they do not know the correct things to say or do not know what questions to ask. Individuals often forget recent events between therapist visits and fail to relay information that could help treatment. Few people have any records or logs that record the person's moods and emotions, and even fewer people can tie those moods and emotions to potential causes and effects. Studies have shown that as people become more depressed, they withdraw from social interactions the times they mostly urgently need the support.

An embodiment of the disclosure seeks to address the aforementioned issues. An embodiment of the disclosure leverages technology to record a user's responses to measurements that are clinically proven to have an impact on mental health. The data can be tracked and analyzed to determine which elements have the greatest impact on an individual's wellbeing. The data can be used to establish a baseline for the individual. When the baseline is established, new data can be used to identify when the individual could use additional support. Unique triggers can be established and identified that will notify predefined contacts that the individual could use additional support. Each contact can be setup for multiple criteria, and different contacts can be associated with different trigger events.

In an example embodiment, when a user experiences issues related to diet, the user's dietician may be notified that the person could use additional support. Further, when a user experiences a low in physical activity, the user's specified workout partner may be notified that the person could use additional support. Further, when a user experiences a depression or low moods, the user's family member or close friend may be notified that the person could use additional support. Further for example, higher-level notifications may contact clinicians or ecclesiastical leaders to notify those persons that the user could use additional support.

The disclosure herein provide means for a user to record data, see historical trends, and see predictions and advice regarding the person's overall wellbeing and mental health. An embodiment of the disclosure automatically reaches out to predefined contacts based on trigger events. The user will no longer be required to directly reach out and ask for help during a difficult time. The user gains more control over the management of personal wellbeing and mental health.

Embodiments of the disclosure provide a quick and simple means to enter metrics for categories that are known to directly impact the user's mental health. The categories may include, for example, the user's stress levels, self-esteem, confidence, energy, exercise, diet, sleep, and others. The categories may be tailored for each user depending on which categories have a greater impact on that user's wellbeing. The user may interact with an application or other user interface to enter a score for each of the categories. In an embodiment, the score is a rating from zero to ten indicating the user's current feeling for that category.

Embodiments of the disclosure provide a quick and simple means for a user to log “boosters.” Boosters include various positive scenarios that are known to improve the user's wellbeing and mental health. Example boosters include performing deep breathing exercises, intentionally engaging in mindfulness, intentionally engaging in good posture, being confident, being kind to others, being heard, performing an act of service, exercising, and others. The boosters, and the value for each booster, may be tailed for each user based on which boosters are known to have the greatest impact on the user's wellbeing. Boosters can improve the user's overall health score for the day, wherein the score provides a numerical representation of the user's wellbeing. Boosters give the user a means to directly impact the overall health score by encouraging the user to engage in an act that will improve the user's wellbeing.

Embodiments of the disclosure automatically reach out to predefined contacts in response to trigger events. In an embodiment, the user defines contacts that should be contacted in response to certain events. For example, the user may indicate that the system should contact the user's workout partner if the user has not completed a workout in a certain number of days. The user may indicate the system should contact the user's spouse, parent, or other close family member if the person's overall health score has been low and the person has been struggling. The user may indicate the system should contact the user's therapist if the user experiences a notable event. The user may indicate the system should contact the user's psychologist or physician if the user appears to be experiencing depression. It should be appreciated that the user or the system may define any number of contacts that should be contact in response to any suitable trigger events.

Embodiments of the disclosure include a reinforcement model to reward a user for tracking metrics. An embodiment uses gamification to apply rewards when a user consistently tracks metrics. In an embodiment, as the user enters data over time, an application tracks the entries and rewards the user with badges or levels. This achievement reward system is focused on the user creating new habits that have a positive impact on the user's life.

Embodiments of the disclosure enable the user to be notified of discounts and offers unique to the user's mental state. These discounts and offers can be based on the user's geographic location. The geographic location of the user may be determined based on the user's mobile telephone device. For example, the user may receive notifications for health food discounts when the user is near stores that may sell items that could improve the user's diet metric. Further for example, the user may receive notifications for fitness opportunities such as gym memberships, local races, public recreation facilities, and so forth that could be used to boost the user's physical activity score. Further examples may be applied to each metric and may be extended to the user's support contact so there is a direct suggestion for an engagement point to further promote interaction between the individuals. For example, the user and one or more of the user's support contacts may be notified of a new public recreation facility, or a new gym, or a new restaurant that could boost the user and the support contact's overall wellbeing.

Embodiments of the disclosure deploy a neural network to analyze past user activity, past community activity, social science data, historical trends, and so forth to predict future highs and lows in the user's wellbeing. For example, a neural network may learn that the user experiences a dip in mental health every winter when the user experiences fewer hours of sunlight. For example, the neural network may learn that the user experiences mental health struggles each year around the anniversary of a family member's death or other tragedy. The neural network may analyze and assess historical information to predict when the user should focus on physical activity, diet, emotional awareness, mindfulness, reaching out to others, and so forth.

Embodiments of the disclosure use multiple technologies to provide a simple approach to tracking and predicting mental health. Embodiments of the disclosure leverage advancements in technology to predict highs and lows in a user's wellbeing. Implementations of the disclosure can be used to change public awareness and clinical research related to mental health.

In the following description of the disclosure, reference is made to the accompanying drawings, which form a part hereof, and in which is shown by way of illustration specific implementations in which the disclosure is may be practiced. It is understood that other implementations may be utilized, and structural changes may be made, without departing from the scope of the disclosure.

The disclosure extends to systems and methods for measuring and recording therapeutics. More specifically, the disclosure extends to systems and methods for providing a measurement and logging service in the mental health field and other fields. The systems and methods may be deployed in a software application for use with any number of computing devices, whether in desktop or mobile form. The application is meant to put control of an individual's overall health and wellbeing into their own hands. Using fun, interactive technology, users can easily navigate the application to find out what elements of their wellbeing are working, and what can be improved upon. Starting with a quick and easy scoring assessment, users can immediately visualize the data, assess how they are doing, and identify people they trust the most, to help them when they need it.

The systems and methods of the disclosure enable personal control over health and self-improvement through engaging visual measurement, built in reinforcement systems, and the incorporation of support networks managed through a fun interactive interface. The systems and methods disclosed integrate a ratings-based algorithm that reflects a user's overall health through a single number called an overall health score. This number provides an immediate snapshot of the individuals wellbeing, which they can then begin to assess and change.

The systems and methods of the disclosure provide a measurement and logging service to various industries, including the mental health industry. However, it will be appreciated that the systems and methods of the disclosure are not limited to the mental health industry as the systems and methods disclosed herein are applicable to many other industries where improvement in performance or in one's ability is sought. It will be appreciated that the disclosure may use the mental health industry as an example of the application of the systems and methods disclosed, but the disclosure extends beyond the scope of the mental health industry into nearly all other industries, particularly where improvement in performance is sought and desirable.

Data is gathered through a user's responses that are submitted through a brief questionnaire each day. The measurement questions are selected from a predetermined bank of questions and will be tailored to each consumer. Over time consumers will only be give questions that have been determined to be the most effective for them. Questions that are not deemed helpful will no longer be asked. So, although all questions will come from a common source pool, not all consumers will be answering the same questions.

Questions are grouped into measurement groups referred to as health metric categories. Each measurement group as well as questions within that group will have a specific weighting that is used to produce an overall health score. In addition to the questions' overall weighting, the algorithm also has the concept of a “half-life” for questions. The more recently a consumer has answered a question, the more weight it has versus the same question from a prior day. By doing this we can present a score at any time using aggregate values from prior days. This smooths out data spikes and provides a better overview of the trending on a how well a consumer is managing their stress or anxiety or managing another aspect for which improvement is sought.

Because each consumer may answer a different set of questions each day, administrators can use the score to track overall health trends for groups of users within the application. Consumers will also identify one or more support contacts. The support contact is like a sponsor in programs such as Alcoholics Anonymous. In the current system and method, it will likely be a trusted friend, family member, co-worker, boss, mental health practitioner, or other professional. The support contact will have the ability to view data about the consumer and receive notifications of trends (good or bad). This will allow them to reach out to the consumer proactively and check on them. Many people with mental health issues are slow to reach out for help directly and this will help ensure they get contacted in potential times of need.

Finally, there is the role of the financial agent. The financial agent will typically be an organization at some level, although it could be a consumer in some revenue models. Examples would be an insurance company, the clinic or hospital at which the practitioner works. Early on the financial agent might be the consumer or the support contact until we gain proper market traction. In most cases the financial agent will not be directly involved in consumer interaction but will monitor trends and aggregate values of multiple users' scores. This data will be used as further justification to continue to pay for the service.

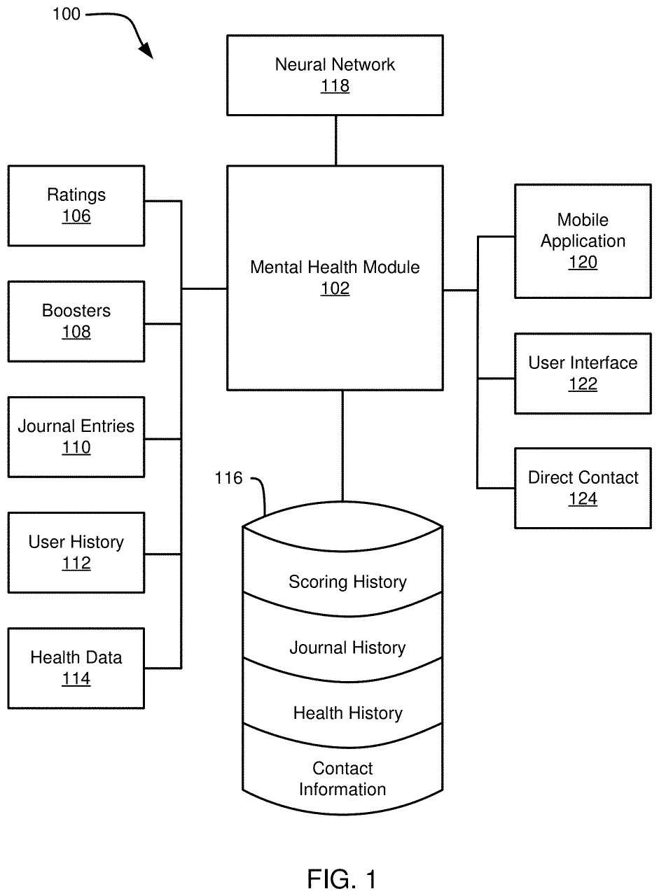

Referring now to the figures, FIG. 1 illustrates an example system 100 for scoring and tracking mental health and acting upon mental health events. The system includes a mental health module 102 for scoring, tracking, predicting, and acting upon mental health information. The mental health module 102 may receive multiple types of information and use that information to predict a user's mental health. In an embodiment, the mental health module 102 uses ratings 106, booster 108, journal entries 110, history 112 information, and health data 114 in calculating mental health scores and predictions. The mental health module 102 may be in communication with multiple output sources, including a mobile application 120, a user interface 122 that may be a web-based user interface, and direct contact 124 with medical professionals or trusted individuals. The mental health module 102 may be in communication with a database 116 storing any suitable data, including scoring history, journal history, health history, contact information, and more. The mental health module 102 may work in connection with a neural network 118 to recognize mental health trends and providing guidance on mental health issues.

In an embodiment, the mental health module 102 further receives data by way of one or more application program interfaces (APIs). The mental health module 102 may receive user-specific data, environmental data, and aggregate data that is deemed relevant for training a neural network or analyzing the user's inputs. In an embodiment, biometric input is received from a sensor. Biometric input includes, for example, heart rate data, sleeping patterns, blood pressure data, and so forth. The mental health module 102 may receive medical data and/or mental health information from the user's personal records. The mental health module 102 may receive calendar appointments and events by way of an API. The mental health module 102 may schedule correlation of events such as treatment date, meetings, and other engagements. Further, the mental health module 102 may associate critical events such as dates of marriage, divorce, birth of a loved one, death of a loved one, beginning a new job, termination from a job, and so forth. The mental health module 102 may receive environmental data from one or more APIs. The environmental data includes, for example, weather data such as temperature, cloud coverage, and precipitation, lunar cycle data, sunrise time, sunset time, hours of daylight per day, UV index, and so forth. The environmental data may further include other data about the user's environment such as traffic data, amount of time for the user to commute to certain locations, and so forth. The mental health module 102 may receive aggregate data by way of an API. The aggregate data includes, for example, seasonal weather trends, regional trends, economic trends, and so forth.

The ratings 106 include metrics entered by a user for different categories. In an embodiment, the user enters a metric from zero to ten for each of multiple categories. Example categories include the user's stress, self-esteem, confidence, energy, exercise, diet, sleep, happiness, physical health, and so forth. Different categories may be presented to different users depending on which categories have the greatest impact on that user's wellbeing and mental health. The metrics for each category may vary for different users or different implementations of the disclosure. In one implementation, the user provides a score for each category ranging from zero to ten or any other suitable numeric range. In an implementation, the user provides a non-numeric response, such as a frowning face, a neutral face, and/or a smiling face to represent the user's feelings for each category. These non-numeric responses can be converted to numeric responses for purposes of determining an overall health score for the user. It should be appreciated that the ratings 106 can be entered in any suitable means and the ratings 106 can be entered for any suitable categories applicable to different users. In an embodiment, a user may enter personalized categories that pertain to that user's individual situation, for example, whether the user is taking prescribed medication or other drugs, the impact of those medications or drugs, whether the person is following a prescribed diet or other diet system, and so forth.

The boosters 108 research-based interventions that, when used or implemented, have a positive effect on mental health. Some boosters 108 are known to have an immediate positive impact on mental health and overall wellbeing. Thoughts, emotions, and behaviors are known to have direct impact on overall mental health and wellbeing. However, behaviors are the easiest to change. Research shows that influencing one of the three will also have a similar effect on the other two. Booster 108 are behavior-based activities. In some embodiments, booster 108 are simple and quick to implement and are effective for positively changing mental health. The boosters 108 provided to a user are determined based on the user's profile, history, and preferences. If some boosters 108 appear to have a greater impact on the user's mental health, then those boosters 108 may rank higher and be located nearer the top on the user interface. Additionally, boosters 108 that are known to have a greater positive impact on a user's wellbeing may have more points assigned thereto. The number of points assigned to a booster 108 dictates how great of an impact that booster 108 will have on the user's overall health score.

In an embodiment, boosters 108 are customized and adjusted as profile and aggregate demographic data is gathered. Boosters 108 may further be customized and adjusted according to historical trends specific to the user or society at large. The listing of boosters 108 may be prioritized according to user preference, frequency of engagement, and targeted correlation to personal rating responses.

In an embodiment, the mental health module 102 provides a certain number of boosters 108 to each user. The number of boosters 108 and the type of boosters 108 may be tailored to each user based on the user's ratings 106, user history 112, health data 114, journal entries 110, and psychological or sociological data. For example, a user with an interest in gardening, or a user living in a climate conducive to gardening, may have a gardening booster 108. Further, the gardening booster 108 may only be available certain times of the year when the user could engage in gardening activities. Further for example, a user with a history playing team sports may have a team sports booster 108. The team sports booster 108 may be provided to the user and may further be provided to other contact persons known to engage in team sports with the user. Further for example, all users may have a service booster 108 for performing service to others or engaging in charitable acts because service is known to have a positive impact on human wellbeing and mental health. The number and identify of boosters 108 provided to each user is dynamic based on the user's current and historical parameters. Additionally, users may create customer boosters 108 tailored to their preferences and identity.

The journal entries 110 are freeform text, image, and/or video entries submitted by the user. The journal entries 110 can be associated with a specific date and timestamp. The overall health score for that date or time when the journal entry was drafted can further be included with the journal entry. The journal entries may include text, videos, images, hyperlinks, messages, emails, and any other suitable files. A user may draft a journal entry to detail what the user did that day, how the user felt that day, what emotions the user experienced, and what had an impact on the user's wellbeing that day.

The journal entries 110 can be scanned by the neural network 118 to identify certain events, circumstances, or people having a significant impact on the user's wellbeing and mental health. In an embodiment, the neural network 118 is trained on a dataset of writings, images, and videos. The neural network 118 can scan the journal entries to identify certain words, phrases, and image content to derive meaning from the journal entry. The journal entry 110 can be scanned by the neural network 118 to determine if the user is experiencing exceptional difficulty and a contact person or healthcare professional should be contacted on behalf of the user. The journal entry 110 can be scanned to identify certain major life events that could have an impact on the user in the future. The journal entries 110 can be scanned to determine tone, state of mind, and mental state of the user to determine what boosters 108 could be especially beneficial to the user's wellbeing at that time.

The user history 112 includes the user's mental health and physical health history. The user history 112 may include clinician notes, medications, allergies, significant life events, and so forth. The user history 112 may further include a log of the user's interaction with the mental health module 102, including the user's responses to ratings 106, boosters 108, journal entries 110, and so forth.

The health data 114 includes important information about the user's mental health and physical health. The health data 114 may include information about the user's physical conditions and the user's dietary and physical activity needs. The health data 114 may include upcoming appointments with healthcare professionals. The health data 114 may include a log of medications currently or previously taken by the user and may further include notations indicating any reactions the user had to those medications. The health data 114 may be retrieved by way of one or more application program interfaces (APIs). The health data 114 may be retrieved from any suitable source, for example, the user's patient medial history, visits with healthcare providers such as physicians, psychologists, or therapists, data from the user's smart watch or other wearable sensor, and data from sensors such as sleep apnea devices and so forth.

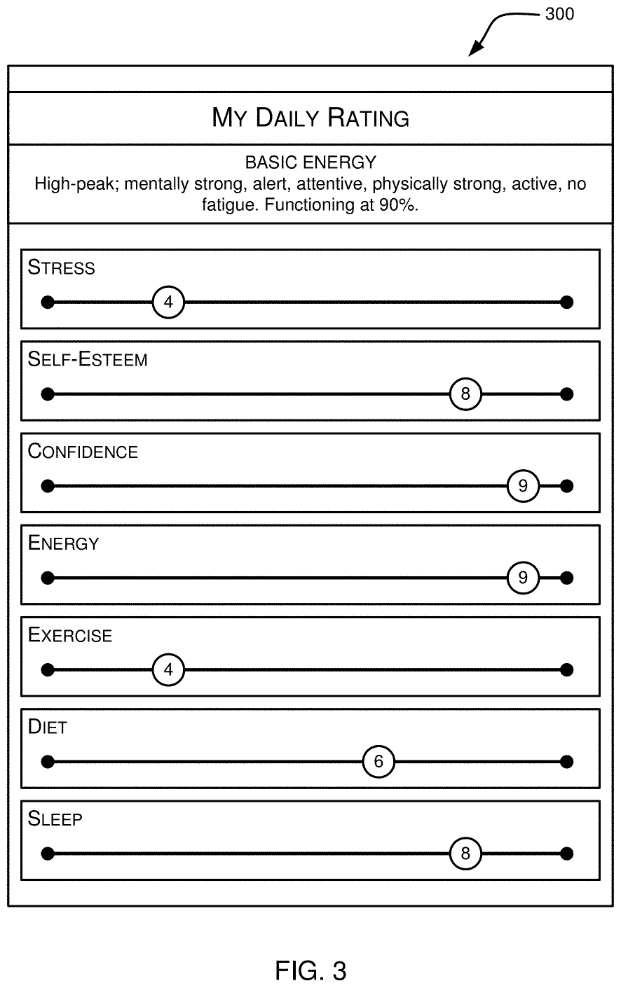

The mobile application 120 and user interface 122 are platforms in which a user may interact with the mental health module 102. Example screenshots of a mobile application 120 are depicted in FIGS. 3-7. The mental health module 102 may be accessible to a user by way of a mobile phone application 120, a user interface 122 on a website, a software platform, and so forth.

The direct contact 124 is a means by which the mental health module 102 can contact or notify one or more persons or entities on the user's contact list. In an embodiment, the mental health module 102 automatically notifies a person or entity on the user's contact list in response to a trigger event. The mental health module 102 may initiate a telephone call with a contact, may send an email or text message to a contact, may mail a letter to a contact, may send a push notification to the contact's account with the mental health module 102, and so forth. In an embodiment, the mental health module 102 initiates an automatic robotic phone call with a contact. In an embodiment, the mental health module 102 initiates an in-person phone call with a contact that is carried out by a person. In an embodiment, the mental health module 102 notifies the user when the mental health module 102 has reached out to one of the contacts on the user's contact list.

In an implementation where the mental health module 102 determines the user may be in danger, the mental health module 102 may automatically notify the user's healthcare profession, may call emergency services, or may notify some other professional. In an embodiment where the mental health module 102 determines the user may be a danger to others, the mental health module 102 may automatically notify the user's healthcare profession, may call emergency services, or may notify some other professional. The mental health module 102 may determine the user is a danger to himself or others based on the user's ratings 106, boosters 108, journal entries 110, user history 112, and health data 114.

The neural network 118 can be trained to scan the user's journal entries 110 to identify issues or significant events occurring in the user's life. The neural network 118 may be trained to scan text of the journal entries 110 and any images or videos associated with the journal entries 110 to determine if the user needs additional support from one of the user's contact persons or a healthcare professional.

The neural network 118 may be trained to analyze and assess the user's history with mental health module 102 to identify trends in the user's lifetime. For example, the neural network 118 may identify that the user struggles with depression each winter when the number of hours sunlight decreases. For example, the neural network 118 may identify that the user is very busy at work and is experiencing a lot of stress. In response, the neural network 118 may provide an indication to the mental health module 102 that the user is undergoing a lot of stress and could use some additional support.

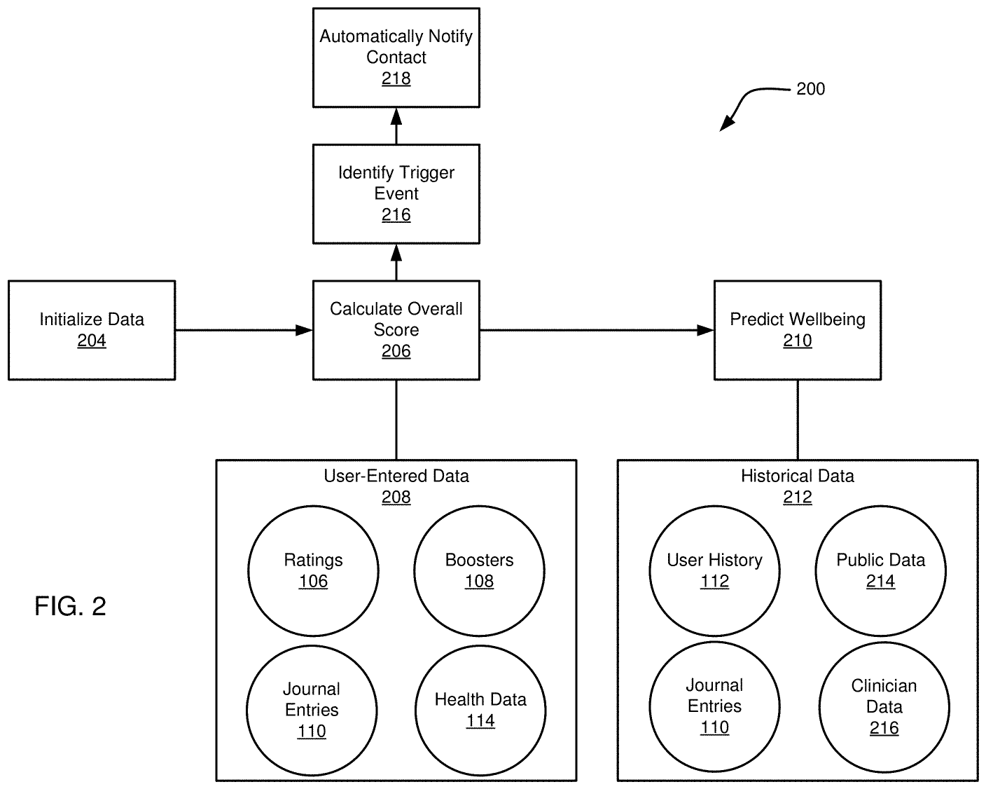

FIG. 2 illustrates a process flow 200 for calculating, analyzing, and predicting a user's wellbeing and mental health. The process flow 200 begins with initializing data at 204. After data is initialized at 204, the system can calculate an overall health score at 206. The overall health score is based on user-entered data 208 for that date or time period. The user-entered data 208 includes ratings 106, boosters 108, journal entries 110, and health data 114. Based on the results of calculating the overall health score 206 and/or calculating additional scoring metrics, the system may identify a trigger event at 216. In response to identifying the trigger event at 216, the system may automatically notify a contact at 218. The system may perform further analytics in addition to calculating the overall health score at 206 by predicting wellbeing at 210. The system may predict wellbeing at 210 using a neural network trained on historical data 212 for mental health and wellbeing. The historical data 212 may include personalized historical data for only that user, may include trend data for a population, may include data associated with sociological and/or psychological studies, and so forth. In an embodiment, the historical data 212 includes user history 112, public data 214, journal entries 110, and clinician data 216.

The data may be initialized at 204 to provide the user a customized set of ratings 106 and boosters 108. The customized ratings 106 options and boosters 108 options may vary day-to-day, hour-to-hour, and so forth. The customized ratings 106 options and boosters 108 options may depend on the user's schedule, current mood, historical trends, and so forth.

In an embodiment, the system is connected with the user's personal event calendar and/or work calendar, and the customized ratings 106 options and boosters 108 options are dependent on the events listed in the user's personal events calendar and/or work calendar. For example, if the user has a busy workday with many meetings, the user may be prompted to engage in good posture, take a moment to meditate, and/or take a moment to engage with colleagues with a positive attitude. These boosters 108 options may be dependent on which boosters are known to have the greatest positive impact on the user's mental health when the user is busy or stressed. Further for example, if the user has a busy workday, then the ratings 106 options may pertain specifically to categories such as stress, feeling overwhelmed, diet, exercise, and so forth.

The overall health score may be calculated at 206 based on current and historical parameters for the user. The parameters may be user-defined or may be identified with a neural network. For example, some parameters may be user-entered data 208 such as ratings 106, booster 108, journal entries 110, and health data 114. Additionally, some parameters may include historical data 212 such as user history 112, public data 214, journal entries 110, and clinician data 216. In an embodiment, a neural network analyzes a user's journal entry 110 to identify an overall mood or emotion for the user based on the language in the journal entry 110. In an embodiment, the overall health score is based only on user-entered data 208 for that date. In an embodiment, the overall health score is based only on user-entered data 208 over a time period. In an embodiment, the overall health score is based on user-entered data 208 and historical data 212 including public data 214.

The overall health score is a single value that represents a snapshot of the user's wellbeing for a given point in time. The overall health score may be a snapshot of the user's wellbeing on a certain date, week, month, moment in time, and so forth. The overall health score is effective for providing the user a meaningful representation of the user's current or historical wellbeing. The overall health score is calculated by aggregating responses to the ratings 106. The overall health score is manipulated to give each rating a unique weight based on the effectiveness and importance of that measurement. The calculations performed to determine the overall health score represent more than the current date and have unique decay rates applied to previous entries to prevent disproportionate valuation of past experiences. By establishing a baseline for an individual, the overall health score can be used throughout the systems and application and across the user's support contacts.

The user's wellbeing may be predicted at 210 by a neural network. The neural network may be trained on historical mental health data for the individual user and/or for larger populations. The neural network may further be configured to analyze the user-entered data 208 in light of public data 214 such as sociological studies and/or psychological studies. The neural network may be trained to predict when the user is most likely to experience a high or low in mental health and overall wellbeing. The neural network may be trained to predict which boosters 108 are most likely to improve the user's mental health.

In an embodiment, the user's wellbeing is predicated at 210 further based on information retrieved from one or more application program interfaces (APIs). The user's wellbeing may be predicated at 210 further based on environmental data, aggregate data, healthcare data, and so forth. Biometric sensor data may be obtained from the user's smart watch or other wearable device. The user's medical and mental health information may be retrieved by way of an API or manually inputted based on the user's personal records. The user's calendar events can be retrieved by way of an API. The user's calendar events may include any suitable events or notifications such as treatment dates, meetings, social engagements, and so forth. The user's wellbeing may further be predicted based on association with critical life events for the user such as dates of marriage, divorce, birth of a loved one, death of a loved one, beginning a new job, being terminated from a job, and so forth.

In an embodiment, the neural network is trained on the user history 112 over time. The neural network may assess trends across the user's historical ratings 106, booster 108, journal entries 110, health data 114, and so forth to identify what events, time periods, or triggers are mostly likely to cause the user to have an increase or decrease in mental health. The neural network may be trained on a large training set of journal entry writings so the neural network can scan the user's journal entries 110 and identify if the user is experiencing a good or poor mental health. The neural network may further be trained to scan the user's journal entries 110 to identify what is impacting the user's mental health, what activities the user has engaged in, how the user is responding to external pressures, and so forth.

In an example, the neural network is trained on a large dataset of journal writings and other writings, so the neural network is trained to scan the user's journal entries 110 and identify the meaning of those journal entries 110. The example neural network may read a user's journal entry 110 indicating that the user's family member passed away at a certain time of year, and that this event is difficult for the user at that time of year. The neural network may then indicate to the system that the user will likely experience mental health struggles at that time of year, every year. The system may respond to this information by preparing the user for the anniversary of the family member's death by tailoring the user's boosters 108 and/or contacting certain persons in the user's contact list.

The trigger event may be identified at 216 in response to the user's ratings 106, boosters 108, journal entries 110, and/or health data 114 for that day or time period. The system may recognize, based on the user-entered data 208, that the user is experiencing some kind of difficulty. The system may then automatically notify a contact at 218 that the user is experiencing a difficulty and could benefit from extra support.

In an example, the trigger event is the user's failure to exercise for a certain time duration. The user may specify this trigger event and indicate that the system should contact the user's workout partner when the user fails to exercise for a certain time period. In an example, the trigger event is the user entering high stress ratings 106 for a period of time. The user may specify this trigger event and indicate that the system should contact the user's friend and suggest a night out or other activity to help relieve the user's stress levels. In an example, the trigger event is the user failing to collect any service-based or charity-based boosters 108 for a time period. The user may specify this trigger event and indicate that the system should reach out to the user's ecclesiastical leader to request a service opportunity for the user. In an example, the trigger event is the user indicating that the user's diet has been poor for a time period. The user may specify this trigger event and indicate that the system should reach out to the user's dietician to request a communication from the dietician, an updated meal plan, and so forth.

In some embodiments, the trigger events are determined by the neural network based on the historical data 212. In an embodiment, when the neural network identifies potential trigger events for the user, and the user meets the requirements for a trigger event, the system may provide a notification to the user indicating that the user could benefit from reaching out to a friend, family member, or other trusted person. Additionally, the system may provide a notification to the user indicating that the user could benefit from engaging in certain activities such as meditation, service work, charity work, mindfulness, exercise, and so forth.

FIG. 3 illustrates an example screenshot 300 of a user interface for collecting ratings 106. The ratings 106 may be collected daily, weekly, hourly, twice per day, and so forth. The ratings 106 may be updated throughout a day, week, month, and so forth. In an embodiment, the system prompts the user to enter ratings 106 at the end of each day. The ratings 106 are customized to each user based on which categories appear to have the greatest impact on the user's wellbeing. Example categories include stress, self-esteem, confidence, energy, exercise, diet, and sleep as shown in FIG. 3. Additional example categories include mindfulness, charity work, anger, mood, traveling, interactions with family, interactions with friends, hobbies specific to the user, and so forth. In an embodiment, the user can specify certain ratings 106 categories and/or the system may provide categories based on which categories appear to have the greatest impact on the user's wellbeing.

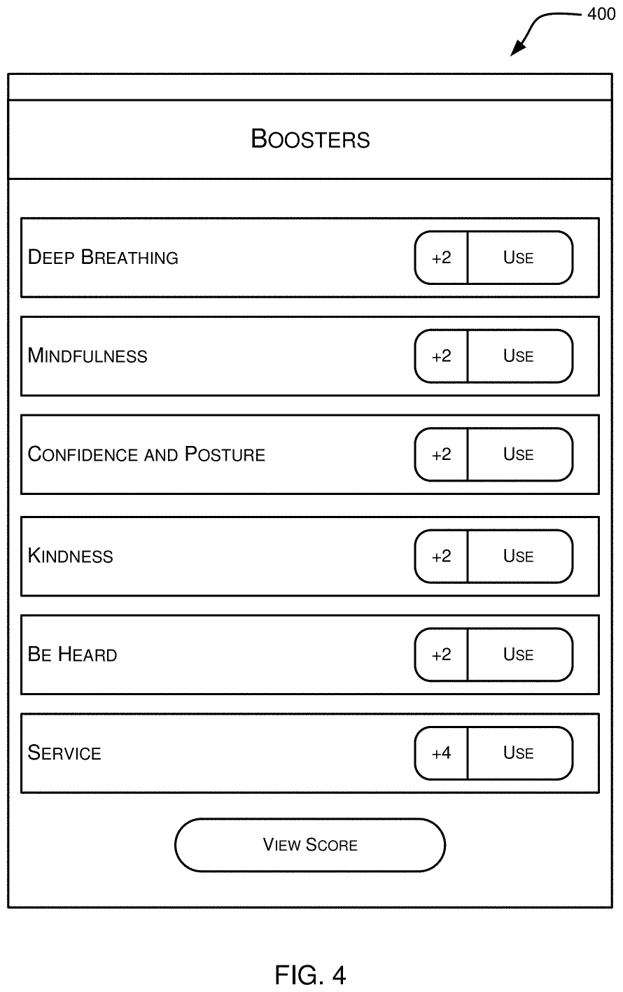

FIG. 4 illustrates an example screenshot 400 of a user interface for collecting booster 108 responses. The boosters 108 may be added at any time and multiple instances of the same booster may be added in one day. In an embodiment, each booster 108 is assigned a point value, and that point value corresponds with the impact the booster 108 will have on the user's overall health score. Example boosters 108 include deep breathing, mindfulness, confidence and posture, kindness, be heard, and service as shown in FIG. 4. Additional example boosters 108 include charity work, smiling, giving a compliment, complimenting yourself, thinking positive thoughts, finishing a project, cleaning a room, speaking with a loved one, and so forth.

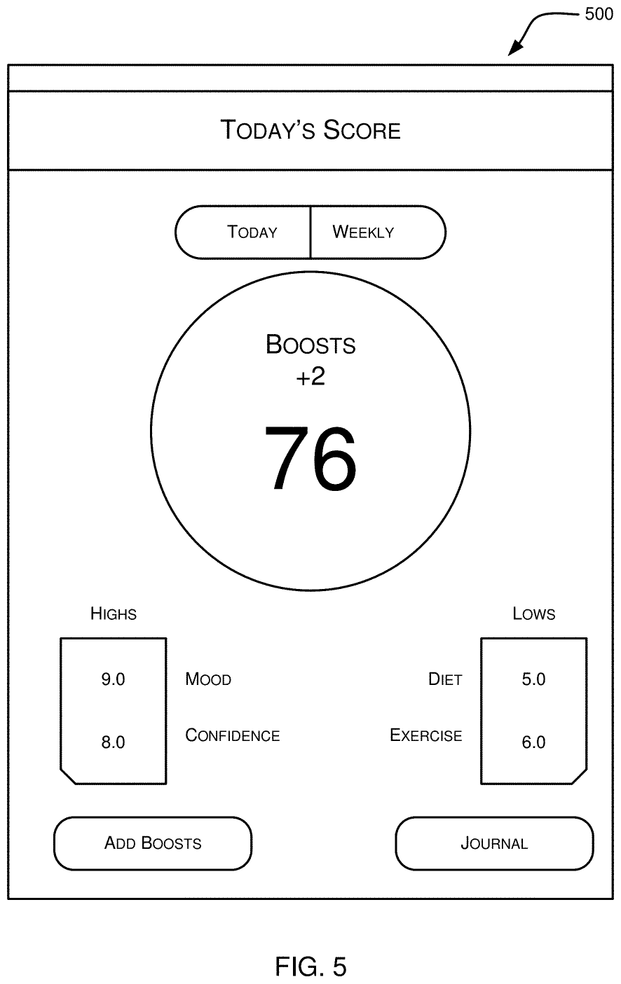

FIG. 5 illustrates an example screenshot 500 of a user interface for providing an overall health score. The overall health score is a snapshot numerical value indicating the user's wellbeing or mental health at a point in time. The overall health score is depending on the user's responses to the ratings 106, the boosters 108, the journal entries 110, and/or the user's health data 114.

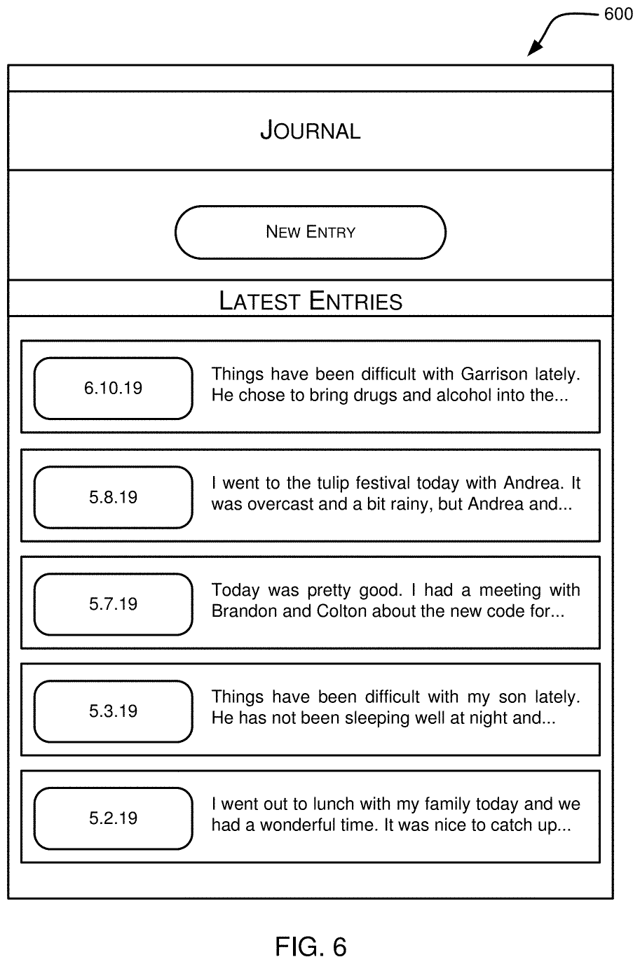

FIG. 6 illustrates an example screenshot 600 of a user interface for collecting journal entries. The screenshot 600 provides a quick summary view of journal entries across a given period. The screenshot 600 presents the journal entries such that a user can scan entries and read the first few lines of text along with associated image thumbnails. In some embodiments, the preview of the journal text is accompanied by the overall health score for that date. The user can filter and search the journal entries based on keywords, date, whether a journal entry is started, the overall health score associated with that journal entry, and so forth.

The journal entries are associated with a certain date and time. Because the journal entries are associated with a certain date, the content of a journal entry can be correlated with metrics to activities that had an impact on the overall health score for that date. User can catalog activities with point-in-time accuracy that will help the user identify the metadata associated with historical scores. This allows the user to read, analyze, and view events that had a significant impact on the user's mental state.

In an embodiment, the mental health module 102 includes a gamification component that provides badges or rewards to the user in response to the user performing booster 108 actions and/or engaging with the mental health module 102.

The mental health module 102 may provide badges to the user. Badges may be grouped into related activities. Groups of badges may be aligned across items such as response measurement and application interaction. Each badge within a group may be progressively more difficult to obtain as the user progresses with the mental health module 102. Levels are an aggregation of badges across the groups of badges so that users can be rewarded for progress with the mental health module 102.

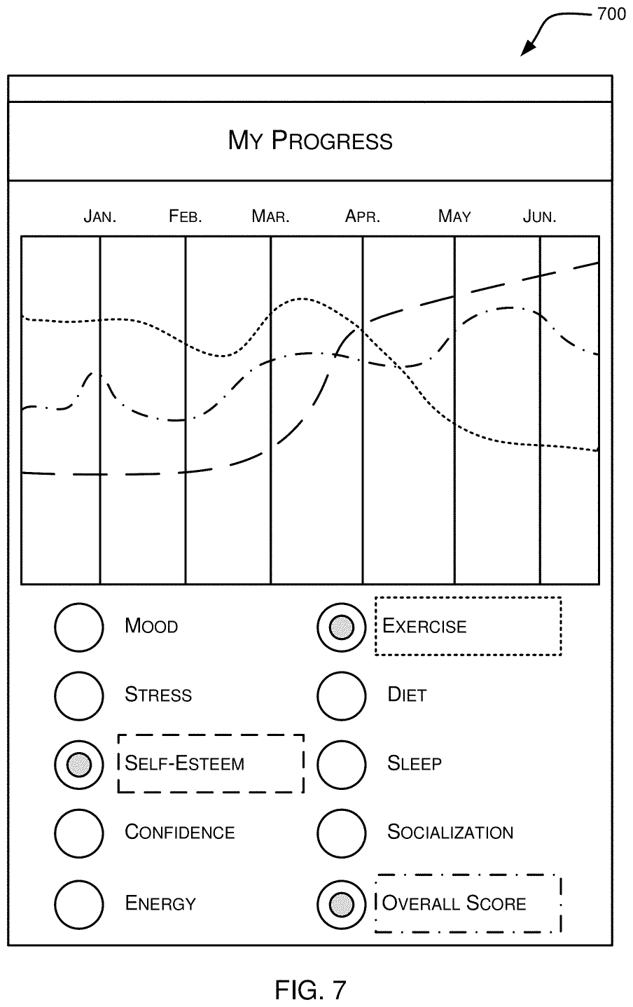

FIG. 7 illustrates an example screenshot 700 of a user interface providing a summary of a user's progress. In the example screenshot 700, the user has interacted with the user interface to have a graph generated over time. The line graph provides information on the user's self-esteem, exercise, and overall health score over time, as shown in the example screenshot 700 in FIG. 7. The user may request any suitable graph that may compare different metrics against each other, may compare metrics over time, may identify which boosters the user has used most frequently or least frequently, which contacts have been notified over time, and so forth. It should be appreciated that the system can generate any suitable reports or graphs that are beneficial to the user in different implementations.

The progress screen gives the user a place to quickly view and compare historical data in a charted form. The user may adjust date ranges to identify trends in the user's wellbeing. The user can quickly overlay different measurements as shown in FIG. 7 to identify correlation patterns with the overall health score. Each measurement will have a different level of impact depending on the user. This allows the user to identify trends in their moods and emotions and allows the user to preemptively take actions that might be needed as the user experiences trials and stressful experiences.

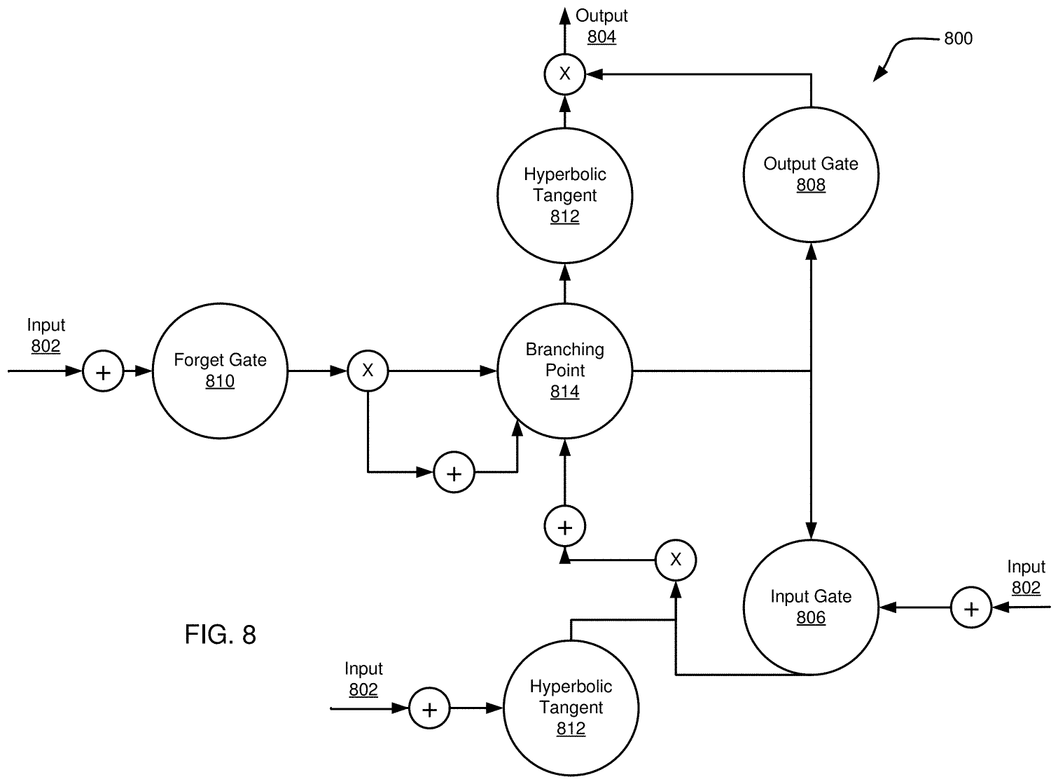

FIG. 8 illustrates a schematic diagram of a long short-term memory network (LSTM network) 800. The LSTM network 800 may be used to analyze journal entries or other datasets to identify patterns where personal or social engagement activities may be triggered. The LSTM network 800 receives input 802 and generates output 804. The LSTM network 800 includes an input gate 806, an output gate 808, a forget gate 810, one or more hyperbolic tangents 812, and a branching point 814. As shown in FIG. 8, the LSTM network 800 can receive input 802 at the input gate 806 and the forget gate 810. The input 802 may include new input and/or recurrent input. In FIG. 8, multiplication calculations are denoted by an “×” and sum calculations are denoted by a “+”.

The LSTM network 800 may be trained initially by source data comprising written entries. The source data may further include images, video streams, and so forth as these data structures may also be present in the journal entries as shown in FIG. 6. The data may be adjusted by backpropagation, weighting, and biases based on correlation with existing user-specific data from daily ratings, environmental data, critical events, and so forth.

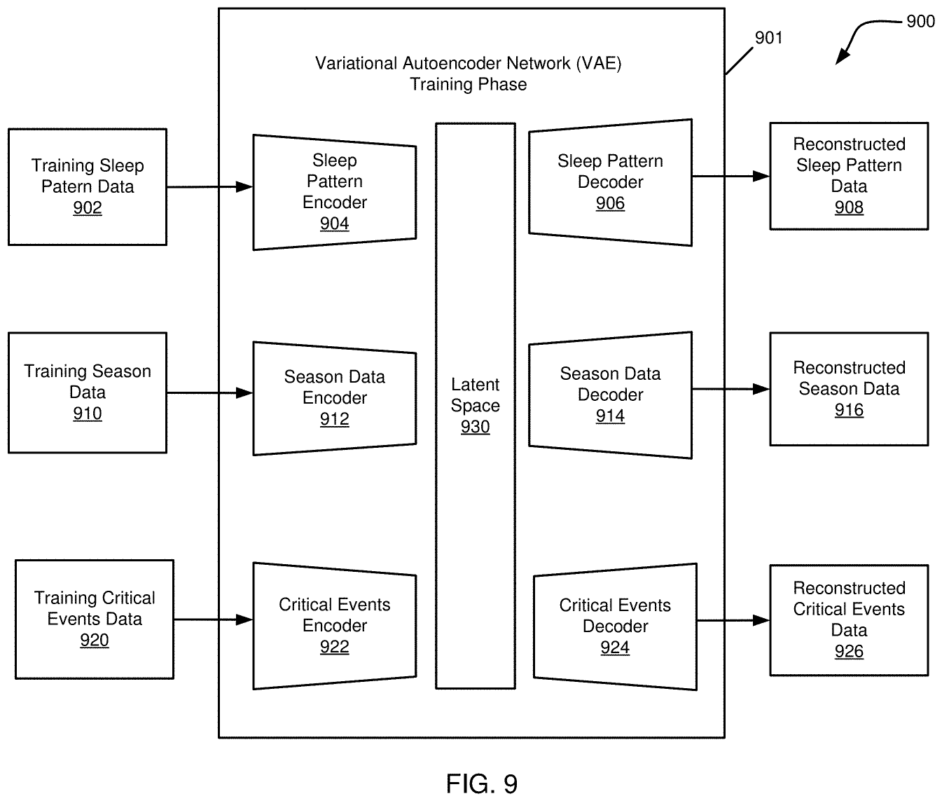

FIG. 9 illustrates a schematic diagram of a training phase 900 of a variational autoencoder network (VAE) 901. In an embodiment, the VAE 901 includes a sleep pattern encoder 904 corresponding with a sleep pattern decoder 906. The VAE 901 may further include a season data encoder 912 corresponding with a season data decoder 914. The VAE 901 may further include a critical events encoder 922 corresponding with a critical events decoder 924. In various embodiments, the VAE 901 may have different encoder-decoder pairs for different datasets. The VAE 901 includes a latent space 930 that is shared by each of the sleep pattern encoder 904, the sleep pattern decoder 906, the season data encoder 912, the season data decoder 914, the critical events encoder 922, and the critical events decoder 924. In an embodiment, each of the sleep pattern decoder 906, the season data decoder 914, and the critical events decoder 924 includes a generative adversarial network (GAN) that may comprise a GAN generator and a GAN discriminator. The VAE 901 receives training sleep pattern data 902 at the sleep pattern encoder 904 and the VAE 901 outputs reconstructed sleep pattern data 908 at the sleep pattern decoder 906. The VAE 901 receives training season data 910 at the season data encoder 912 and outputs reconstructed season data 916 at the season data decoder 914. The VAE 901 receives training critical events data 920 at the critical events encoder 922 and outputs reconstructed critical events data 926 at the critical events decoder 924.

In further embodiments, the VAE 901 may include an encoder-decoder, may receive training data for, and may output reconstructed data for multiple different datasets. Other datasets include, for example, daily ratings, journal entries, support contact notification and/or response, heart rate, blood pressure, sleep patterns, temperature, precipitation, season of the year, hours of light per day, correlation to critical events, and so forth. A dataset pertaining to daily ratings may be combine with other datasets to correlate highs and lows in daily ratings with other events, such as a decreased number of hours of daylight, an anniversary of a critical event in the user's life, decreased sleep, and so forth. Physical metrics such as heart rate, blood pressure, sleep patterns, temperature, and precipitation may be gathered automatically from a sensor such as a heart rate monitor, a smart watch, and so forth. These physical metrics may further be analyzed to identify correlations between, for example, high blood pressure and increased pressure at work for the user.

The VAE 901 may serve as central machinery for analyzing and assessing input data received from the user. In an embodiment, the VAE 901 is trained under the assumption that the input training data is sampled at the same time. Under this assumption, the VAE 901 may be trained to assess the entirety of the input data to identify correlations between, for example, physical metrics such as heart rate and blood pressure, seasonal metrics such as weather or the number of hours of daylight each day, critical events in the user's life, daily ratings, and so forth. In an embodiment, the VAE 901 is trained on a complete dataset for a single period in the user's life. The complete training dataset may include the training sleep pattern data 902, the training season data 910, and the training critical events data 920 as shown in FIG. 9 and may further include other training datasets such as training daily ratings data, training journal entry data, training support contact notification and response data, training heart rate data, training blood pressure data, training body temperature data, training precipitation data, and so forth. It should be appreciated the training phase 900 as shown in FIG. 9 is illustrative only and the training dataset may be tailored to specific uses of the systems and methods disclosed herein. After the VAE 901 is trained, the VAE 901 can take as input actual user data for a period of time and analyze that data to identify correlations between the user's different metrics. The VAE 901 may receive real-time physical data from a heart monitor or other device and may further receive user-inputted data such as daily ratings and journal entries.

In an embodiment, the encoder and the decoder in each encoder-decoder pair are adversarial to one another and are configured to generate reconstructed data. The encoder is configured to receive a training dataset and map the training dataset to a compress latent representation in the latent space 930. The decoder may include a GAN having a GAN generator and a GAN discriminator. The decoder is configured to decode the compressed latent representation from the latent space 930. The GAN of the decoder may be configured to generate the reconstructed dataset.

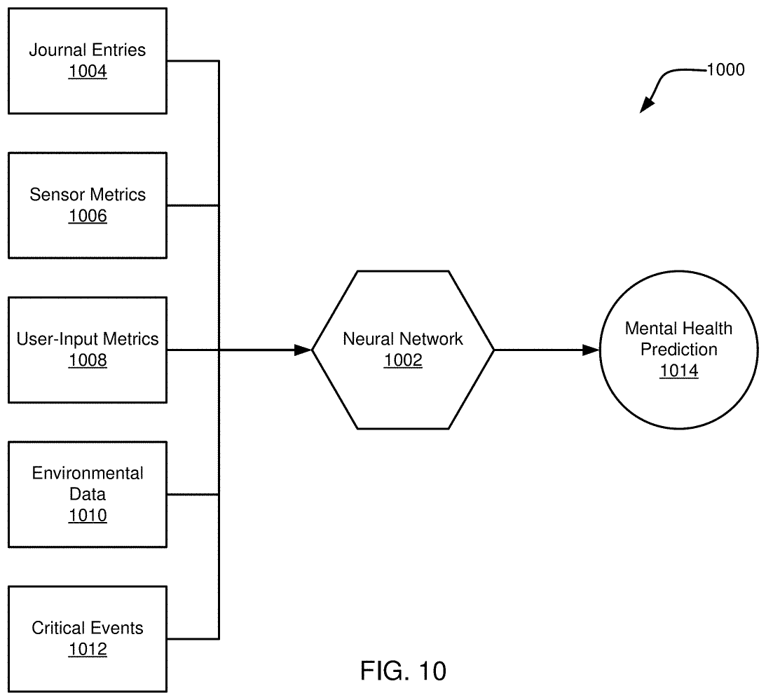

FIG. 10 illustrates a schematic block diagram of a process flow 1000 for analyzing and assessing information with a neural network 1002. The process flow 1000 includes analysis by a neural network 1002 to generated mental health predictions 1014. The neural network 1002 receives multiple datasets for a single period of time and performs analysis on those datasets. Example datasets include journal entries 1004, sensor metrics 1006, user-input metrics 1008, environmental data 1010, and critical events 1012. The neural network 1002 may be trained to identify correlations or patterns within the input datasets to generate a mental health prediction 1014.

The journal entries 1004 include user-input journal entries as discussed herein. The neural network 1002 may receive the raw journal entries 1004 or may receive an assessment on the journal entries as determined by, for example, a LSTM network 800. The journal entries 1004 may include text, image, and video data, and may further include hyperlinks or pointers to other data. The journal entries 1004 may be analyzed to identify events, social triggers, psychological triggers, and other pertinent data based on the user's entries. For example, the journal entries 1004 may be assessed to determine that an anniversary of a critical life event for the user is upcoming. The date of that critical life event may be logged for future use in generating future mental health predictions 1014.

The sensor metrics 1006 may include metrics from, for example, a heart rate monitor, a blood pressure monitor, a sleep monitor, a brain wave activity monitor, an imaging sensor, and so forth. The sensor metrics 1006 may be updated periodically by a healthcare provider. The sensor metrics 1006 may be updated and received in real-time by a sensor worn by the user, such as a heart rate monitor. The sensor metrics 1006 may be retrieved from a healthcare provider by way of an application program interface (API) that enables the system to automatically retrieve updated metrics based on permissions granted by the user and the healthcare provider.

The user-input metrics 1008 include metrics input by the user such as daily ratings, boosters, and so forth. The current user-input metrics 1008 for a certain time period may be assessed along with historical user-input metrics 1008 for other time periods.

The environmental data 1010 includes information about the user's environment. This may include environment data such as weather data, number of hours of daylight per day, and so forth. This may further include other pertinent information about the user's environment such as traffic conditions, amount of time for the user to commute to work or other locations, the distance the user lives from important locations, and so forth.

The critical events 1012 includes information and a log of critical events 1012 in the user's life. For example, the critical events 1012 may include an indication of the date when a close family member of the user passed away, a date when the user was married or divorce, a date when the user first became sober from drugs or alcohol, or any other date that is deemed important or critical in the user's life. The critical events 1012 may be manually inputted by the user or a healthcare provider. The critical events 1012 may be identified by a contact person of the user. The critical events 1012 may be identified based on the user's journal entries 1004. The timing of critical events 1012 may be assessed to identify correlations between critical events or anniversaries of critical events and highs or lows in the user's other metrics.

The neural network 1002 outputs the mental health prediction 1014 based on one or more of the datasets. The mental health prediction 1014 may include an indication that the user is likely to experience a drop in mental health and may benefit from additional support from contact people, healthcare providers, and so forth. The mental health prediction 1014 may be provided to the user by way of a notification to remind the user to prepare for an upcoming struggle or other issue.

For example, if the neural network 1002 determines that the user suffers from depression brought on by a decrease in the number of hours of sunlight per day, the mental health prediction 1014 may be a notification to the user indicating that the user should prepare for a decrease in sunlight and should make an effort to seek out ultraviolet radiation through natural or artificial sources. For example, if the neural network 1002 determines that the user suffers from a downturn in mental health on the anniversary of the death of the user's loved one, the mental health prediction 1014 may include one or more notifications to the user's contact person indicating that the anniversary of the loved one's death is upcoming and the user could benefit from additional support. For example, if the neural network 1002 determines that the user experiences a spike in heart rate and/or blood pressure in response to increased stress at work, the mental health prediction 1014 may include a notification to the user to take time to relax or medicate to take control of the user's physical manifestations of stress. The mental health prediction 1014 could further include a notification to the user's healthcare provider indicating that the user is experienced increased heart rate and blood pressure. This notification may be sent in response to a trigger event, for example, the user's heart rate and/or blood pressure exceeding threshold levels for a time period.

In an embodiment, the neural network 1002 is a variational autoencoder. The neural network 1002 may work in connection with or may further include a long short-term memory network. The neural network 1002 may be any suitable neural network known in the art or developed.

The neural network 1002 may function by forward propagation and backward propagation of training data in input data. The neural network 102 may be configured with parameters such as weights and biases guiding the analysis of input data. The neural network 1002 may take a set of training data that may include one or more of journal entries 1004, sensor metrics 1006, user-input metrics 1008, environmental data 1010, and critical events. The training data may be processed by the neural network 1002 to generate a mental health prediction 1014. These mental health predictions 1014 may be obtained with values of expected labels to calculate loss. The neural network 1002 performs backpropagation to propagate this loss to each of the parameters making up the model of the neural network 1002. The propagated information is used to update the parameters of the neural network with the gradient descent in a way that the total loss is reduced, and a better model is obtained. This process may be continually iterated until a good model for the neural network 1002 is achieved.

In an embodiment, the mental health prediction 1014 is a value that represents the state of the user's mental health or overall wellbeing. The mental health prediction 1014 may indicate a likelihood the user needs or could benefit from contact with external real-world sources of support. Such external real-world sources of support include for example, family members, friends, listed contact persons, healthcare professionals, ecclesiastical leaders, and so forth.

The neural network 1002 is implemented in real-time to determine the necessity of a trigger event. A trigger event causes the system to notify a contact person that the user would benefit from additional support. In an embodiment, the neural network 1002 is implemented continually throughout the data submission process to produce aggregate data used at a sponsor level to evaluate the general wellbeing of a group such as a company wellness program or an insurance company's evaluation of the quality and result of treatment programs.

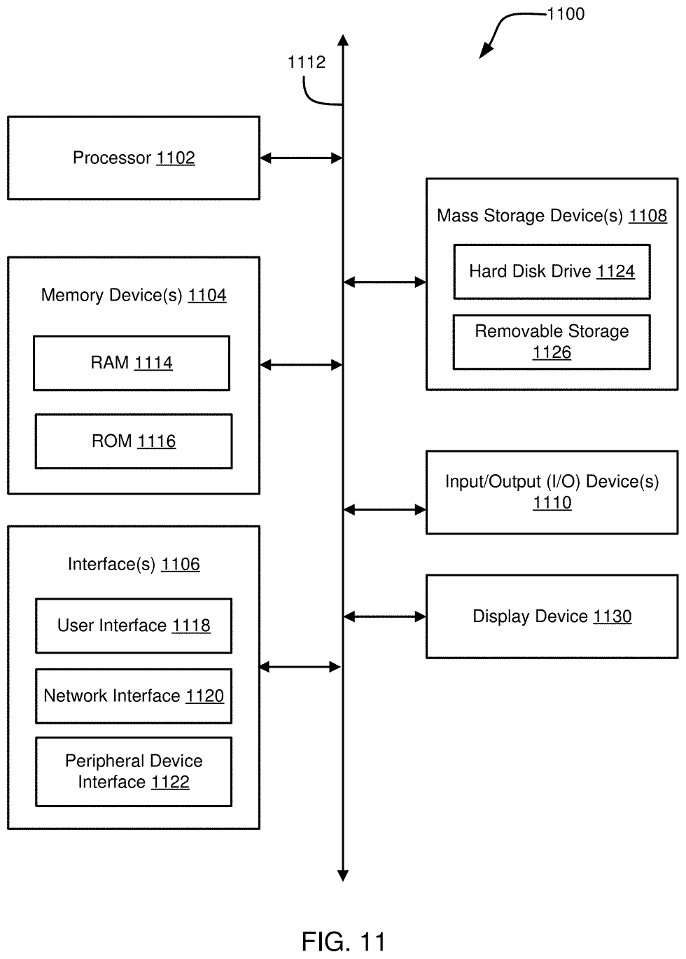

Referring now to FIG. 11, a block diagram of an example computing device 1100 is illustrated. Computing device 1100 may be used to perform various procedures, such as those discussed herein. In one embodiment, the computing device 1100 can function as a mental health module 102, a neural network 118, or the like. Computing device 1100 can perform various monitoring functions as discussed herein, and can execute one or more application programs, such as the application programs or functionality described herein. Computing device 800 can be any of a wide variety of computing devices, such as a desktop computer, in-dash computer, vehicle control system, a notebook computer, a server computer, a handheld computer, tablet computer and the like.

Computing device 1100 includes one or more processor(s) 1102, one or more memory device(s) 1104, one or more interface(s) 1106, one or more mass storage device(s) 1108, one or more Input/output (I/O) device(s) 1110, and a display device 1130 all of which are coupled to a bus 1112. Processor(s) 1102 include one or more processors or controllers that execute instructions stored in memory device(s) 1104 and/or mass storage device(s) 1108. Processor(s) 1102 may also include various types of computer-readable media, such as cache memory.

Memory device(s) 1104 include various computer-readable media, such as volatile memory (e.g., random access memory (RAM) 1114) and/or nonvolatile memory (e.g., read-only memory (ROM) 1116). Memory device(s) 1104 may also include rewritable ROM, such as Flash memory.

Mass storage device(s) 1108 include various computer readable media, such as magnetic tapes, magnetic disks, optical disks, solid-state memory (e.g., Flash memory), and so forth. As shown in FIG. 11, a particular mass storage device is a hard disk drive 1124. Various drives may also be included in mass storage device(s) 1108 to enable reading from and/or writing to the various computer readable media. Mass storage device(s) 1108 include removable media 1126 and/or non-removable media.

Input/output (I/O) device(s) 1110 include various devices that allow data and/or other information to be input to or retrieved from computing device 1100. Example I/O device(s) 1110 include cursor control devices, keyboards, keypads, microphones, monitors or other display devices, speakers, printers, network interface cards, modems, and the like.

Display device 1130 includes any type of device capable of displaying information to one or more users of computing device 1100. Examples of display device 1130 include a monitor, display terminal, video projection device, and the like.

Interface(s) 1106 include various interfaces that allow computing device 1100 to interact with other systems, devices, or computing environments. Example interface(s) 1106 may include any number of different network interfaces 1120, such as interfaces to local area networks (LANs), wide area networks (WANs), wireless networks, and the Internet. Other interface(s) include user interface 1118 and peripheral device interface 1122. The interface(s) 1106 may also include one or more user interface elements 1118. The interface(s) 1106 may also include one or more peripheral interfaces such as interfaces for printers, pointing devices (mice, track pad, or any suitable user interface now known to those of ordinary skill in the field, or later discovered), keyboards, and the like.

Bus 1112 allows processor(s) 1102, memory device(s) 1104, interface(s) 1106, mass storage device(s) 1108, and I/O device(s) 1110 to communicate with one another, as well as other devices or components coupled to bus 1112. Bus 1112 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1100 and are executed by processor(s) 1102. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, if any, any future claims submitted here and in different applications, and their equivalents.

What is claimed is:

1. A method comprising:
receiving a plurality of user ratings responsive to health metric categories;
receiving a journal entry comprising one or more of text, images, or videos;
calculating an overall health score for the user based at least in part on the plurality of user ratings;
storing a plurality of overall health scores for the user over time;
assessing the plurality of overall health scores for the user over time to identify a correlation between at least one health metric category and a positive or negative change in the overall health score for the user; and
personalizing the health metric categories provided to the user based on which health metric categories have the greatest correlation with a positive or negative change in the overall health score for the user.

2. The method of claim 1, further comprising processing at least one of the plurality of user ratings or the journal entry with a neural network to predict a future positive or negative change in the overall health score for the user.

3. The method of claim 1, further comprising training a neural network to predict a future positive or negative change in the overall health score for the user based on the journal entry, wherein training the neural network comprises training with a dataset comprising text entries or images.

4. The method of claim 1, wherein calculating the overall health score for the user comprises:
aggregating the plurality of user ratings responsive to the health metric categories; and
weighting each health metric category associated with the plurality of user ratings based on a correlation between each health metric category and a positive or negative change in the overall health score for the user.

5. The method of claim 4, wherein calculating the overall health score for the user further comprises:
calculating a time decay rate for each of the health metric categories based on a correlation between each health metric category and a positive or negative change in the overall health score for the user; and
applying the corresponding time decay rate to each of the plurality of user ratings to adjust for an impact of each of the plurality of user ratings on the overall health score for the user over time.

6. The method of claim 1, further comprising receiving an indication the user performed a booster action, and wherein calculating the overall health score for the user comprises calculating further based on the user performing the booster action.

7. The method of claim 6, further comprising:
assessing booster actions performed by the user over time to identify a correlation between at least one type of booster action and a positive or negative change in the overall health score for the user; and
personalizing booster action suggestion provided to the user based on which booster actions performed by the user have the greatest correlation with a positive change in the overall health score for the user.

8. The method of claim 1, further comprising:
identifying a most positive health metric category associated with a highest user rating for a time period;
identifying a most negative health metric category associated with a lowest user rating for a time period; and
providing the most positive health metric category and the most negative health metric category to the user.

9. The method of claim 1, further comprising assessing at least one of the overall health score or the plurality of user ratings to determine whether the user satisfies a trigger event.

10. The method of claim 1, further comprising providing a notification to a contact in response to the user satisfying the trigger event.

11. A system comprising:
a mental health module in communication with a user interface and a database storing a plurality of user inputs;
a neural network in communication with the mental health module;
the mental health module comprising one or more processors configurable to execute instructions stored in non-transitory computer readable storage media, the instructions comprising:
receiving a plurality of user ratings responsive to health metric categories;
receiving a journal entry comprising one or more of text, images, or videos;
calculating an overall health score for the user based at least in part on the plurality of user ratings;
storing a plurality of overall health scores for the user over time;
assessing the plurality of overall health scores for the user over time to identify a correlation between at least one health metric category and a positive or negative change in the overall health score for the user; and
personalizing the health metric categories provided to the user based on which health metric categories have the greatest correlation with a positive or negative change in the overall health score for the user.

12. The system of claim 11, wherein the neural network further comprises one or more processors configurable to execute instructions stored in non-transitory computer readable storage media, instructions for the neural network comprises processing at least one of the plurality of user ratings or the journal entry to predict a future positive or negative change in the overall health score for the user.

13. The system of claim 11, wherein the instructions cause the one or more processors of the mental health module to calculate the overall health score for the user by:
aggregating the plurality of user ratings responsive to the health metric categories; and
weighting each health metric category associated with the plurality of user ratings based on a correlation between each health metric category and a positive or negative change in the overall health score for the user.

14. The system of claim 13, wherein the instructions cause the one or more processors of the mental health module to calculate the overall health score for the user further by:
calculating a time decay rate for each of the health metric categories based on a correlation between each health metric category and a positive or negative change in the overall health score for the user; and
applying the corresponding time decay rate to each of the plurality of user ratings to adjust for an impact of each of the plurality of user ratings on the overall health score for the user over time.

15. The system of claim 11, wherein the instructions further comprise:
assessing booster actions performed by the user over time to identify a correlation between at least one type of booster action and a positive or negative change in the overall health score for the user;
and personalizing booster action suggestion provided to the user based on which booster actions performed by the user have the greatest correlation with a positive change in the overall health score for the user.

16. Non-transitory computer readable storage media storing instructions that, when executed by one or more processors, cause the one or more processors to:
receive a plurality of user ratings responsive to health metric categories;
receive a journal entry comprising one or more of text, images, or videos;
calculate an overall health score for the user based at least in part on the plurality of user ratings;
store a plurality of overall health scores for the user over time;
assess the plurality of overall health scores for the user over time to identify a correlation between at least one health metric category and a positive or negative change in the overall health score for the user; and
personalize the health metric categories provided to the user based on which health metric categories have the greatest correlation with a positive or negative change in the overall health score for the user.

17. The non-transitory computer readable storage media of claim 16, wherein the instructions further cause the one or more processors to:
train a neural network to predict a future positive or negative change in the overall health score for the user based on the journal entry using a dataset comprising text entries or images; and
process at least one of the plurality of user ratings or the journal entry with the neural network to predict a future positive or negative change in the overall health score for the user.

18. The non-transitory computer readable storage media of claim 11, wherein the instructions cause the one or more processors to calculate the overall health score for the user by:
aggregating the plurality of user ratings responsive to the health metric categories;
weighting each health metric category associated with the plurality of user ratings based on a correlation between each health metric category and a positive or negative change in the overall health score for the user;
calculating a time decay rate for each of the health metric categories based on a correlation between each health metric category and a positive or negative change in the overall health score for the user; and
applying the corresponding time decay rate to each of the plurality of user ratings to adjust for an impact of each of the plurality of user ratings on the overall health score for the user over time.

19. The non-transitory computer readable storage media of claim 11, wherein the instructions further cause the one or more processors to receive an indication the user performed a booster action, and wherein calculating the overall health score for the user comprises calculating further based on the user performing the booster action.

20. The non-transitory computer readable storage media of claim 19, wherein the instructions further cause the one or more processors to:
assess booster actions performed by the user over time to identify a correlation between at least one type of booster action and a positive or negative change in the overall health score for the user; and
personalize booster action suggestion provided to the user based on which booster actions performed by the user have the greatest correlation with a positive change in the overall health score for the user.
